(19) United States
(12) Patent Application Publication
(10) Pub. No.: US 2017/0340490 A1
(43) Pub. Date: Nov. 30, 2017

(54) UNDERGARMENT

(71) Applicant: OLGA HERNANDEZ, FALLS CHURCH, VA (US)

(72) Inventor: OLGA HERNANDEZ, FALLS CHURCH, VA (US)

(21) Appl. No.: 15/168,172

(22) Filed: May 30, 2016

(51) Int. Cl. A61F 13/72; A41B 9/04; A61F 13/66; A61F 13/84

(52) U.S. Cl. CPC A61F 13/72; A61F 13/665; A41B 9/04; A61F 2013/8497

(57) ABSTRACT

An undergarment/underwear including at least one discreet mark indicating the appropriate location to place an undergarment pad in order to avoid leaks and/or discharge.

The invention relates to an'tauter garment for properly placing an undergarment pad to avoid leaks. Today's menstrual period products and incontinent products focus on undergarment pads lacking understanding of several factors affecting the leak displacement.

### SUMMARY

The present disclosure resolves the problem of individuals with periodic vaginal periods who have difficulty placing an undergarment pad. This problem may arise due to several factors such as first time user due to hormonal changes, weight lost, weight gain and others. The individual faces the challenge that the flow may have changed and the undergarment may be stained due to the misplacement a the undergarment pad. It is the objective of this invention to avoid leaks from the undergarment pad by providing an undergarment indicating at least one location where to allocate the undergarment pad on the undergarment using a discreet mark. In order to determine where to place the location of the undergarment discreet mark, at least one of the following factors is taken in consideration: weight, height leak flow, age, undergarment type, pad type, body type and individual posture.

The undergarment discreet mark is camouflaged with undergarment print and/or decoration. Furthermore, the discreet mark can be one hidden until warm heat or sound is applied to the fabric or textile wherein the at least one discreet marking placed acting as a water mark. Moreover, the discreet mark can be implemented in a different way like using techniques such as dye sublimination, heat transfer, sewing, printing and others known in the art or future development of fabric/textile decoration.

In order to decide the place wherein the discreet mark is going to be placed, certain characteristics must be considered before and during the manufacturing of the undergarment. Characteristic such as size, design type, discreet mark type and others are fundamental to determine the outcome of the undergarment. Therefore, it is another objective of this invention to protect the undergarment and make it last longer in good standing conditions based on the aforementioned.

### BRIEF DESCRIPTION OF THE DRAWINGS

FIG. 1 shows a prior art undergarment.

FIG. 2 shows an undergarment partial top view with a discreet front mark.

FIG. 3 shows an undergarment front view with a front discreet mark.

FIG. 4 shows an undergarment back view with two discreet marks.

FIG. 5 shows show an option of camouflage the undergarment discreet mark.

FIG. 6 shows an undergarment partial top view with a discreet back mark.

FIG. 7 shows an undergarment pad placed according to the undergarment front discreet mark.

### DETAILED DESCRIPTION

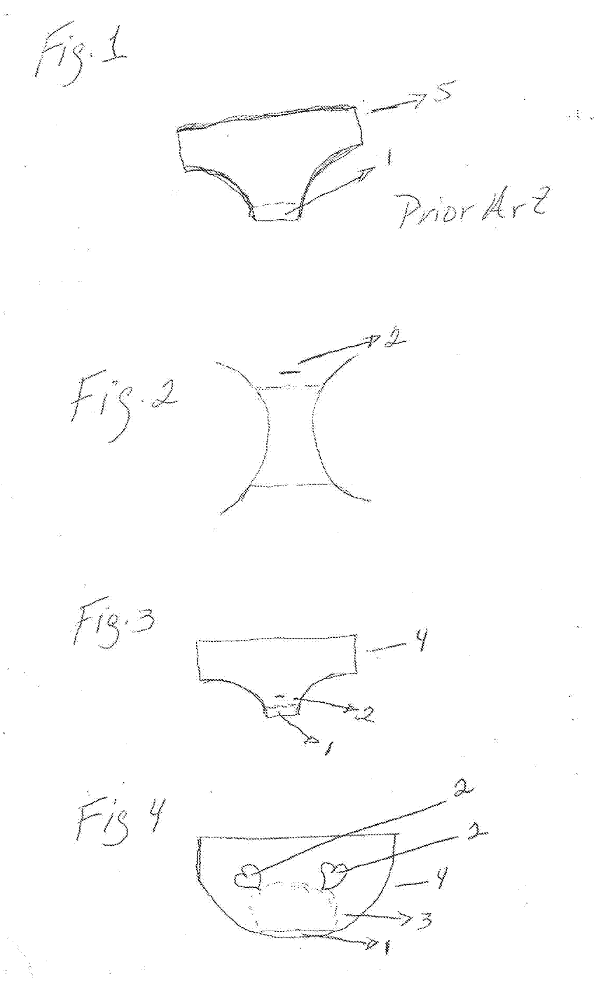

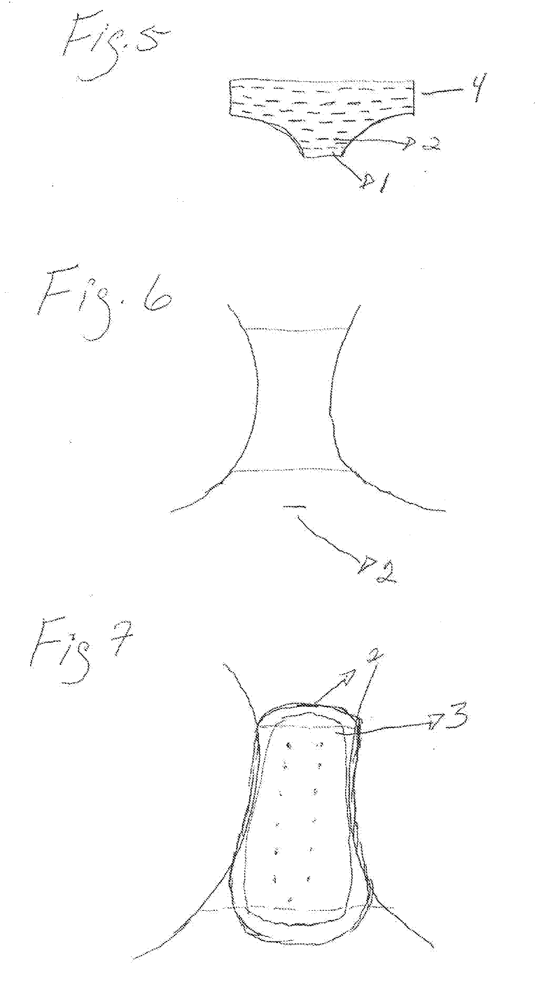

The present disclosure shows how an undergarment discreetly aids users to property determine the use of an undergarment pad 3 in order to avoid leaks and protect the underwear/undergarment. FIG. 1 shows a prior art, which is a common known undergarment 5 with its respective undergarment lining 1. FIG. 2 shows a discreet mark 2 above the lining of the undergarment 4 front side from the top as seen by the user. FIG. 6 shows an alternative option; contrary to FIG. 2, the discreet mark 2 is located on the back of the undergarment 4. The discreet mark 2 indicates where the top edge of the undergarment pad 3 should be placed according to at least one characteristic of weight, height, leak flow, age, undergarment type, pad type, body type, individual's posture, math equation and a combination thereof in the front and/or back of the undergarment 4. The same characteristics and/or conditions can be applied when at least one discreet mark 2 is on the back of the undergarment 4 as shown in FIG. 4.

FIG. 5 shows an example of many ways of how at least one discreet mark 2 can be made discreetly simulated, camouflage and/or hidden by several ways including, but not limited to using the same pattern, shape, slightly discoloration, increase in hue color and/or combination thereof and/or shape of the discreet mark 2 in the undergarment 4 as a repetitive, consecutive and/or decorative icon/mark. The use of water mark technology can also be used to represent the at least one discreet mark. FIG. 4 shows two heart icons wherein the bottom closing angle of a heart is used to indicate the location where the edge of the undergarment pad 3 should be placed on the back of the undergarment 4, While FIG. 4 shows the at least one discreet mark 2 being a heart, any other shape such as a start, triangles, others as well as a combination of different shapes can be used in lieu of the heart icon/mark shown in FIG. 4.

FIG. 3 shows a front view of the undergarment 4 with at least one discreet mark 2 above the undergarment lining 1.

FIG. 7 shows the edge of the undergarment pad 3 being placed the based on the discreet mask 2 indication. While FIG. 7 shows the edge of the undergarment pad 3 to be used, the discreet mark 2 is not limited to the edge of the undergarment pad 3. The undergarment pad 3 is made with several layers, which can be seen through from the top. Any of these layers and/or a combination of layers can be used with slightly alteration to the determination where to place the discreet mark 2 as there is a correlation between the undergarment pad 3 and undergarment type.

While this disclosure specifies hack and front sides of the undergarment 4; it is understood by those skilled in the art that determination of exchanging characteristics of the at least one discreet mark 2 from front and back sides of the aforementioned disclosure can be performed with some modifications of the variables evaluated due to the asymmetrical shapes.

It is understood that various modifications and variations could be made thereto by those skilled in the art without departing from the scope of the invention. Therefore, it is to be understood that within the scope of the claims, the invention may be practiced in another way than described herein.

What is claimed is:

1. An undergarment comprising:
at least one discreet mark;
wherein the at least one discreet mark is presented on at least one side of the undergarment.

2. The undergarment according to claim 1, further comprises the at least one discreet mark being made of at least one of the following: yarn, fabric, textile, dye, ink, bleach, wax, glue, stamp, paint, plastic, metal, and a combination thereof.

3. The undergarment according to claim 1, further comprises the at least one discreet mark is determined based on at least one of the following: weight, height, leak flow, age, undergarment type, pad type, body type, individual's posture, math equation and a combination thereof.

4. The undergarment according to claim 1, further comprises the at least one discreet mark can be noticed by applying heat.

5. The wider garment according to claim 1, further comprises a packaging stating the criteria of the discreet mark.

6. The undergarment according to claim 4, wherein the applying heat is based on an organism touch.

7. The undergarment according to claim 1, further comprises the at least one discreet mark can be noticed by applying sound, vibration and/or noise.

8. The undergarment according to claim 1, further comprises the at least one discreet mark is a water mark.

9. The undergarment according to claim 1, further comprises the at least one discreet mark is camouflaged with the undergarment decoration.

10. The undergarment according to claim 1, further comprises the at least one discreet mark location varies based upon the different layers of the undergarment pad.

11. The undergarment according to claim 1, further comprises the at least one discreet mark contains at least one of increase hue color and slightly decoloration of a repeated pattern.

12. The ink according to claim 2 is invisible ink.

13. The math equation according to claim 3 changes based upon decision of at least one predetermined characteristic of a user.

14. An undergarment comprising:
at least one discreet mark;
wherein the at least one discreet mark is based upon a decision of at least one predetermined characteristic of a user.

15. The undergarment according to claim 14, further comprises the at least one discreet mark is camouflaged with the undergarment decoration.

16. The undergarment according to claim 14, further comprises the at least one discreet mark location varies based upon the different layers of the undergarment pad.

17. The undergarment according to claim 14, further comprises the at least one discreet mark is determined based on at least one of the following: weight, height, leak flow, age, undergarment type, pad type, body type, individual's posture, math equation and a combination thereof.

18. The undergarment according to claim 14, further comprises the at least one discreet mark being made of at least one of the following: yarn, fabric, textile, dye, ink, bleach, wax, glue, stamp, paint, plastic, metal and a combination thereof.
